(19) United States
(12) Patent Application Publication
(10) Pub. No.: US 2017/0296113 A1
(43) Pub. Date: Oct. 19, 2017

(54) Combined device that measures the body weight and balance index

(71) Applicant: Hong Kong Baptist University, Hong Kong (HK)

(72) Inventors: Jeffrey Taikin CHEUNG, Hong Kong (HK); Xinan LI, Hong Kong (HK); Hongyuan XU, Hong Kong (HK)

(21) Appl. No.: 15/485,244

(22) Filed: Apr. 12, 2017

Related U.S. Application Data
(63) Continuation-in-part of application No. 15/276,798, filed on Sep. 27, 2016.
(60) Provisional application No. 62/322,397, filed on Apr. 14, 2016.

(51) Int. Cl. A61B 5/00; A61B 5/11; G01G 19/44

(52) U.S. Cl. CPC A61B 5/4023; A61B 5/6887; A61B 5/7275; A61B 5/1116; A61B 5/1122; A61B 5/112; G01G 19/44

(57) ABSTRACT

The present invention relates to an apparatus for measuring the balance ability; postural deviation, reflex and fall risk assessment of a human subject. In particular, the present invention comprises of an elastic support mounted platform with dynamic response to external motion stimuli, a IMU and data analysis software processing device. The dynamic response from the platform also simulates realistic experience in moving over rough terrain with different topography.

### CROSS-REFERENCE TO RELATED APPLICATIONS

This application claims priority from U.S. Provisional Patent Application Ser. No. 62/322,397 filed on Apr. 14, 2016 and is a continuation-in-part of U.S. Non-Provisional patent application Ser. No. 15/276,798 filed on Sep. 27, 2016, which is a continuation-in-part of U.S. Non-Provisional patent application Ser. No. 14/622,933 filed on Feb. 16, 2015, which claims the benefit of U.S. Provisional Patent Application Ser. No. 61/940,801 filed on Feb. 17, 2014. The disclosures of U.S. Provisional Patent Application Ser. No. 62/322,397, U.S. Non-Provisional patent application Ser. No. 15/276,798, U.S. Non-Provisional patent application Ser. No. 14/622,933 and U.S. Provisional Patent Application Ser. No. 61/940,801 are hereby incorporated by reference in their entirety.

### FIELD OF INVENTION

The present invention relates to method and apparatus for measuring balance ability; postural deviation and fall risk assessment of a human subject. In particular, the present invention provides dynamic stimuli to test the balance ability of the subject and measures the subject's balance ability without visual sensory input of the subject.

### BACKGROUND OF INVENTION

Human balance index is a key health indicator. It requires constant postural adjustment controlled by neuro-motor interaction based on visual, vestibular and proprioception sensory input in order to maintain the center of gravity (COG) of an individual within a base support. Balance is affected when any one part of the complex chain of events does not function properly. Aside from the intrinsic factors, other factors such as intoxication due to alcohol and drug use as well as other physical conditions, such as sickness and aging, also play a significant role.

There are two classes of traditional methods to assess balance: functional assessment and physiological assessment. Functional assessment tests require patient to perform a set of predesigned physical tasks and the results are scored based on the performance. Typical examples include: Fugl-Meyer Test (Fugl-Meyer A R., Jaasko L., Olsson S., Steglind S., The Post-Stroke Hemiplegic Patient: A Method for Evaluation of Physical Performance, Scand, H, Reh. Med., 7 (1975)), Barthel Index measurement (Mahoney F., Barthel D., Functional Evaluation: The Barthel Index, Maryland State Med. J., 1, 14 (1965)) and Berg Test (Berg K O., Maki B E., Williams J I., Holliday P J., Wood-Dauphinee S L., Clinical and Laboratory Measures of Postural Balance in an Elderly Population, Arch. Phys. Med. Rehabil., 73, pp1073-1088 (1992).) These tests are commonly used in clinical practice as they are quick to obtain results and also expensive equipment is not necessary. However, they lack quantitative information.

The second type is physiological assessment that measures the subject's center of gravity (COG) movement while maintaining a balancing stance. There are number of different approaches, however, only a few are available commercially while the remainders are mainly used in laboratory and research environment.

One of the earliest physiological assessment measurements is the use of a potentiometric displacement transducer to measure the anterior-posterior movement of a subject (Fernie G., Holiday P., Postural Sway in Amputees and Normal Subjects, The Journal of Bone and Joint Surgery, 60-A, pp 895, (1978).) Other approaches also include the use of mechanical devices such as the rotations of a perforated wheel attached to subject's body; Sway Magnetometers; insoles embedded with polymer pressure sensor and video imager combined with force platform. However, all these devices have very limited use as they have low resolution and are invasive in nature. Currently, most of the commercial as well as research balance measurement devices use force plate array placed on a static platform or position sensors placed on an unstable platform.

In a force plate based device, a multiple number of force plates are embedded at different location on the static platform. The location of the downward projection of the COG of the subject is determined by the load distribution among the force plates. The system displays the planar trajectory of COG movement as the subject's body shifts constantly to keep balance. During the test, the subject must watch the COG location which is displayed on the monitor screen and shift the body to move the COG either toward the center as close as possible within a given time duration or from one location to another designated location. The position accuracy and the amount of eclipsed time are used to calculate balance index. These devices are bulky and have long measurement time from 20 seconds to over one minute. During data acquisition, the test subject must keep eyes open to view the COG movement on the display monitor, therefore the contribution of vestibular and proprioceptive sensory systems on balance cannot be studied in an isolated way. Finally, the COG is determined only by the movement in the horizontal plane, while the movements in the vertical direction are not accounted for.

Another existing method is described in US Patent Nos.: U.S. Pat. No. 5,627,327A and U.S. Pat. No. 5,830,158A. These devices use an unstable platform on a convex rocker with the test subject standing on the top flat surface to keep balance. As the subject is trying to gain balance, the body movement will cause the platform to rock about both orthogonal axes of the horizontal plane. The amount of movement is recorded with inclinometers and the data is used to determine the balance index. Again, these devices are very bulky and only record the lateral shift of the subject's COG.

It is the objective of the present invention to provide a simplistic apparatus for measuring balance ability; postural deviation and fall risk assessment of a human subject.

Citation or identification of any reference in this section or any other section of this application shall not be construed as an admission that such reference is available as prior art for the present application.

### SUMMARY OF INVENTION

The objective of the present invention is to provide method and apparatus for measuring balance ability; postural deviation and fall risk assessment of a human subject. The present invention comprises an elastic support mounted platform with dynamic response to external motion stimuli, an inertial measurement unit (IMU) and a data analysis computing device. The present invention requires short measurement time, is simple to operate and is compact in size. The present invention can be combined with one or more other devices such as a regular bathroom weight scale or a balance exercise pad for use as a multi-functional personal health monitoring device. In one embodiment., the present apparatus is combined with a high density elastomer foam pad specifically used for balance exercise; therefore a single device can have dual function as an exercise pad and a balance monitoring device. The present device is fundamentally different from existing balance monitoring equipment that uses a set of force plate to detect the shift of the center of gravity (COG) of the subject while trying to keep balance. The existing force plate based balance monitoring device requires the user to shift his COG and visualize it moving along a prescribed path on a static platform. On the other hand, the platform of the present invention is unstable and wobbly which provides dynamic stimuli to the user to keep balance. This configuration allows for the monitoring of balance without visual sensory input of the subject, therefore balance ability by non-visual sensory input of the subject, such as vestibular and proprioceptive input, can be measured independently. In addition, the dynamic response from the platform also simulates realistic experience in moving over different topographies (such as rough terrains). This feature can also create interactive action for gaming and balance training.

In a first aspect of the present invention, there is provided a method for measuring a balance index of a human subject comprising providing a flat platform mounted on one or more elastic supports to provide a wobbly surface for the subject to stand on, the platform comprises an inertial measurement unit (IMU); placing said subject on the flat wobbly platform, measuring center of gravity (COG) of said subject while the subject balances on the platform, measuring movements of COG of said human subject in three orthogonal vectors over a time interval to obtain a COG trajectory in a three dimension space over the time interval, summing over the COG trajectory of said human subject in three dimension space to obtain a gait energy of said movements over said time interval; dividing said gait energy over said time interval to obtain a gait power value, and normalizing said gait power value to determine as a measure of the balance index for said human subject.

In a first embodiment of the first aspect of the present invention, there is provided a method for measuring balance anisotropy and assessing risk of falling for a human subject comprising placing the subject to stand on the unstable flat platform and measuring relative magnitude of balance index in four orthogonal directions of said human subject while the subject balances on said unstable platform. The four orthogonal directions consist of front facing direction, back direction, left direction and right direction of said human subject.

In a second embodiment of the first aspect of the present invention, there is provided a method for measuring postural deviation of a human subject comprising measuring deviation of said human subject's COG trajectory on the unstable platform. The deviation of the subject's COG trajectory is the deviation of trajectory of COG when the subject is standing on the flat platform from the trajectory of the COG of an unloaded flat platform.

In a third embodiment of the first aspect of the present invention, there is provided a method for measuring balance index, balance anisotropy, fall risk assessment and postural deviation of a human subject, wherein the elastic supports are selected from compression springs, extension springs, leaf springs, magnetic repulsion, elastomer balls and cylinders, closed cell high density elastomer foam cushion, inflated rubber ball, inflated rubber cushion, floats on fluid, suspended platform or a combination thereof.

In a fourth embodiment of the first aspect of the present invention, there is provided a method for measuring balance index, balance anisotropy, fall risk assessment and postural deviation of a human subject wherein said platform is a closed cell high density elastomer foam pad and the IMU is mounted on a rigid base plate at the center of said platform, wherein said rigid base plate is connected to at least one rigid longitudinal body that radiate from the center of the rigid base plate.

In a fifth embodiment of the first aspect of the present invention, there is provided a method for measuring balance index, balance anisotropy, fall risk assessment and postural deviation of a human subject wherein said platform further comprises arrays of cavities inside to further provide instability and increase the detection sensitivity.

In a sixth embodiment of the first aspect of the present invention, there is provided a method for measuring balance index, balance anisotropy, fall risk assessment and postural deviation of a human subject, wherein the IMU, the rigid base plate and the at least one rigid longitudinal body are embedded inside the platform.

In a seventh embodiment of the first aspect of the present invention, there is provided a method for measuring balance index, balance anisotropy, fall risk assessment and postural deviation of a human subject, wherein the IMU is a tri-axial accelerometer.

In a second aspect of the present invention, there is provided an apparatus for measuring a balance index of a human subject comprising a flat platform mounted on one or more elastic supports to provide a wobbly surface for the human subject to stand on. Said flat platform comprises an inertia measurement unit that measures acceleration in all three orthogonal directions. The one or more elastic supports are selected from compression springs, extension springs, leaf springs, magnetic repulsion, elastomer balls and cylinders, closed cell high density elastomer foam cushion, inflated rubber ball, inflated rubber cushion, floats on fluid, suspended platform and a combination thereof.

In a first embodiment of the second aspect of the present invention, there is provided an apparatus for measuring balance index, balance anisotropy, fall risk assessment and postural deviation of a human subject comprising a flat platform having a platform mounted on elastic supports to provide a wobbly surface for the subject to stand on and an inertial measurement unit (IMU) to measure the acceleration in all three orthogonal directions, wherein the elastic supports are selected from compression springs, extension springs, leaf springs, magnetic repulsion, elastomer balls and cylinders, elastomer foam cushion, inflated rubber ball, inflated rubber cushion, floats on fluid, suspended platform and any combination thereof.

In a second embodiment of the second aspect of the present invention, there is provided an apparatus for measuring balance index, balance anisotropy, fall risk assessment and postural deviation of a human subject, wherein said platform is a close cell high density elastomer foam pad and the IMU is mounted on a rigid base plate at the center of said foam pad, wherein said base plate is connected to at least one rigid longitudinal body that radiate from the center of the base plate.

In a third embodiment of the second aspect of the present invention, there is provided an apparatus for measuring balance index, balance anisotropy, fall risk assessment and postural deviation of a human subject, wherein the IMU, the rigid base plate and the at least one rigid longitudinal body are embedded inside the platform.

In a fourth embodiment of the second aspect of the present invention, there is provided an apparatus for measuring balance index, balance anisotropy, fall risk assessment and postural deviation of a human subject, wherein the IMU is a tri-axial accelerometer.

In a fifth embodiment of the second aspect of the present invention, there is provided an apparatus for measuring balance index, balance anisotropy, fall risk assessment and postural deviation of a human subject further comprising a computing device. The computing device or a processor is connected to the flat platform to store and process the balance index, balance anisotropy, fall risk assessment and postural deviation measurements.

In a sixth embodiment of the second aspect of the present invention, there is provided an apparatus for measuring balance index, balance anisotropy, fall risk assessment and postural deviation of a human subject, wherein the computing device is connected to the flat platform via wired or wireless data connection.

Throughout this specification, unless the context requires otherwise, the word “include” or “comprise” or variations such as “includes” or “comprises” or “comprising”, will be understood to imply the inclusion of a stated integer or group of integers but not the exclusion of any other integer or group of integers. It is also noted that in this disclosure and particularly in the claims and/or paragraphs, terms such as “included”, “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the present invention.

Furthermore, throughout the specification and claims, unless the context requires otherwise, the word “include” or variations such as “includes” or “including”, will be understood to imply the inclusion of a stated integer or group of integers but not the exclusion of any other integer or group of integers.

Other definitions for selected terms used herein may be found within the detailed description of the present invention and apply throughout. Unless otherwise defined, all other technical terms used herein have the same meaning as commonly understood to one of ordinary skill in the art to which the present invention belongs.

Other aspects and advantages of the present invention will be apparent to those skilled in the art from a review of the ensuing description.

### BRIEF DESCRIPTION OF DRAWINGS

The above and other objects and features of the present invention will become apparent from the following description of the present invention, when taken in conjunction with the accompanying drawings, in which:

FIG. 1 shows the schematic of one embodiment of the apparatus of the present invention to measure balance index. The apparatus comprises two platforms connected via elastic spacers. An Inertial Measurement Unit (IMU) is embedded in the top platform to record acceleration in all three axes.

FIG. 2 shows schematic side view of one embodiment of the apparatus of the present invention.

FIG. 3 shows the schematic of one embodiment of the apparatus of the present invention that measures balance index and body weight.

FIG. 4a shows the top view schematics of one configuration of the IMU in one embodiment of the apparatus of the present invention.

FIG. 4b shows the side view of one configuration of the IMU in one embodiment of the apparatus of the present invention.

FIG. 4c shows the side view of one configuration of the IMU in one embodiment of the apparatus of the present invention, where IMU is buried inside the platform, facing proximal to the surface where the test subject stands.

FIG. 4d shows the side view of one configuration of the IMU in one embodiment of the apparatus of the present invention, where IMU is buried inside the platform, facing distal from the surface where the test subject stands.

FIG. 5a shows the output signal of an IMU placed on the surface of the foam with rigid longitudinal bodies as described in FIG. 4b when a person is standing on it while trying to keep balance.

FIG. 5b shows the output signal of an IMU placed on the surface of the foam without rigid longitudinal body when a person is standing on it while trying to keep balance.

FIG. 6a shows an example of trajectory of COG in acceleration space of a human subject in 3-D representation X and Y are axes in the horizontal plane and Z is the axis perpendicular to the horizontal plane.

FIG. 6b shows examples of 2-D representation of the trajectory of COG of a human subject in acceleration space. X and Y are axes in the horizontal plane and Z is the axis perpendicular to the horizontal plane.

FIG. 7 shows the relationship between raw data acceleration vectors \vec{Aᵢ}\vec{Aᵢ₊₁}, \vec{Aᵢ₊₂} and gait force vector \vec{A_{Gi}}and \vec{A_{Gi+1}}.

FIG. 8 shows the correlation of balance index measured with Biodex Balance System SD and balance index measured by the apparatus of the present invention. Two sets of data are normalized at the data point shown.

FIG. 9 shows prediction of fall probability and direction. The heave dashed line arrows are the average acceleration vectors in the quadrant.

FIG. 10 shows the postural deviation obtained from balance measurement.

FIG. 11 shows the recovery time of two individuals after initial imbalance excitation.

FIG. 12 shows the dependence of balance index measured by the apparatus of the present apparatus on age and the effect of visual (eyes open) and vestibular sensory (eyes closed) input.

### DETAILED DESCRIPTION OF THE INVENTION

The presently claimed invention is further illustrated by the following experiments or embodiments which should be understood that the subject matters disclosed in the experiments or embodiments may only be used for illustrative purpose but are not intended to limit the scope of the presently claimed invention:

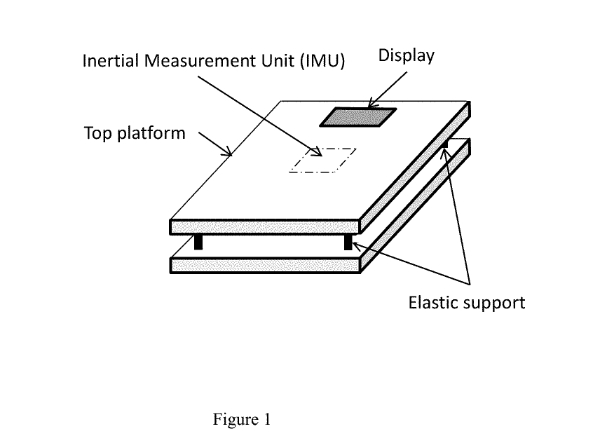

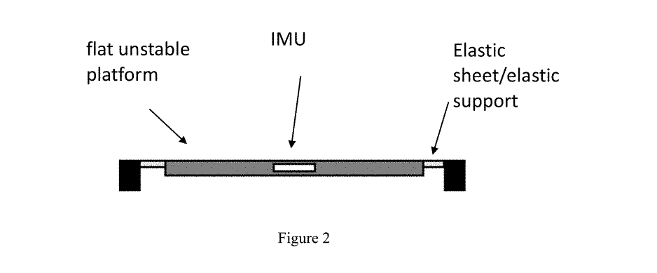

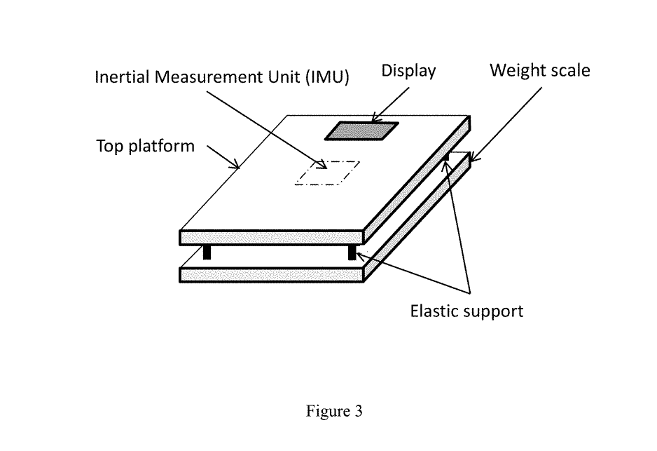

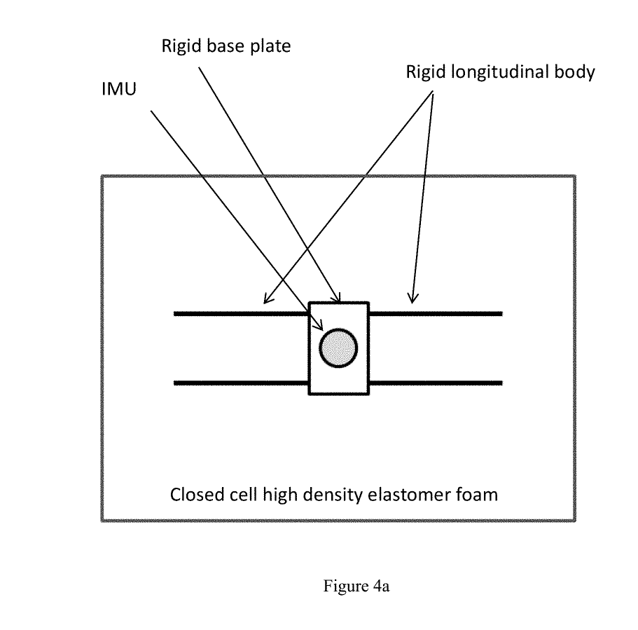

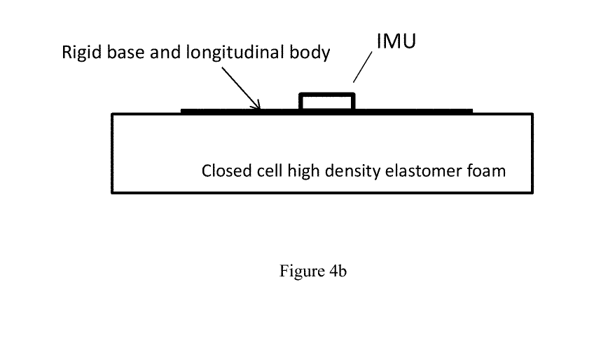

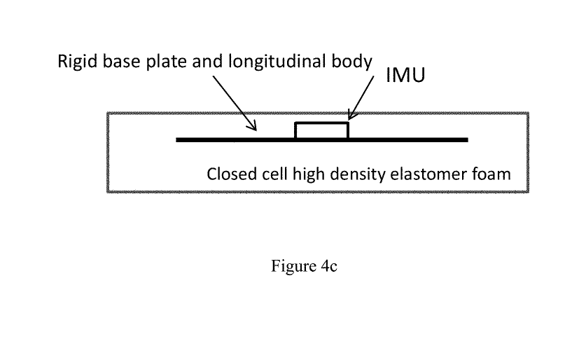

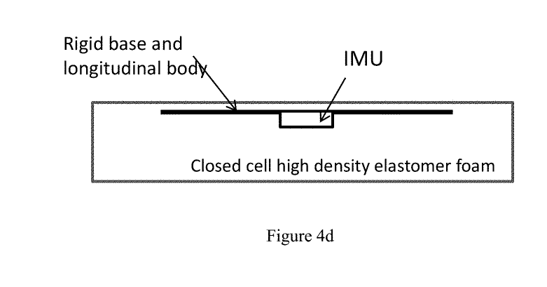

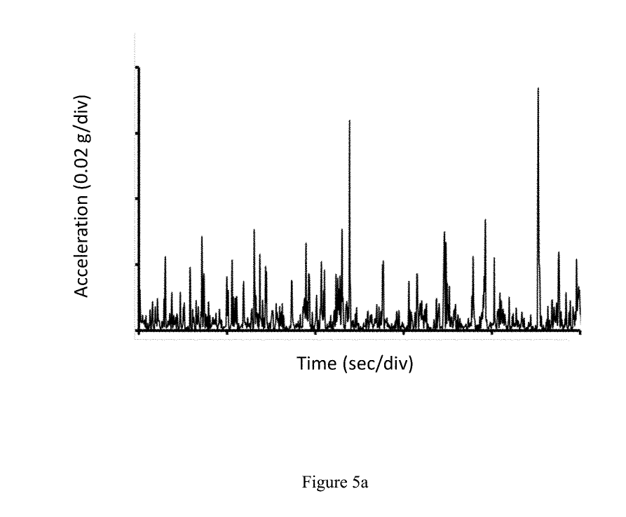

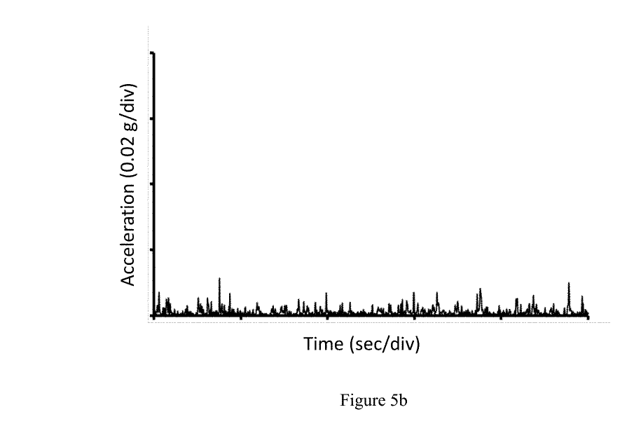

This invention addresses the problems in size, measurement time, the limitation of having visual sensor input during test and the lack of dynamic interaction during test. The apparatus of the present invention comprises a flat platform mounted on one or more elastic supports to provide unstable wobbly surface and an IMU to measure acceleration in all three directions. Schematic design of one embodiment of the apparatus of the present invention is shown in FIG. 1. There is no specific limitation to the types of the elastic supports. The elastic supports can be any supports that provide an unstable wobbly surface as desired by one skilled in the art. Examples of elastic supports include, but are not limited to, compression springs, extension springs, leaf springs, magnetic repulsion, elastomer balls and cylinders, elastomer foam cushion, inflated rubber ball, inflated rubber cushion, floats on fluid, suspended platform or any combination thereof. In order to reduce the size and the height of the apparatus, FIG. 2 shows another embodiment of the present apparatus. Two opposite sides of the flat platform are individually attached to two solid rigid bodies via elastomeric sheet as the elastic support to provide dynamic spring-like movement of the platform. The present apparatus further comprises other devices such as a bathroom weight scale, as shown in FIG. 3, to provide multi-functional health monitoring. The strength of the elastic support mounted base can be changed to adjust the apparatus' sensitivity to adapt to different users for optimum performance. FIGS. 4a-4d shows different configuration of flat platform of the present apparatus. In one embodiment, the flat platform is made of high density elastic foam pad which is used as a platform for physical training to improve balance. The flat platform may be made of other elastic or inelastic materials as desired by one skilled in the art. FIG. 4a illustrates one embodiment of the present apparatus where the elastomer foam flat platform having a rigid base plate and one or more rigid longitudinal body attached to the rigid plate, and IMU is situated on the rigid plate. The longitudinal body may be rods or tubes. FIG. 4b shows the top view of the embodiment of FIG. 4a. FIG. 4c illustrates another embodiment of the present apparatus where the IMU, rigid base plate and longitudinal bodies are embedded inside the elastomer flat platform. FIG. 4d illustrates yet another embodiment of the present apparatus where the IMU, rigid base plate and longitudinal bodies are embedded inside the elastomer flat platform. As seen in FIGS. 4c and 4d, orientation of the rigid base plate and IMU can be arranged differently. IMU can be arranged proximal to the surface on which the test subject stands (FIG. 4c) or distal to the surface on which the test subject stands (FIG. 4d). The closed cell high density elastomer foam with super soft quality to offer destabilizing properties. To further improve its destabilizing sensitivity, the flat platform further comprises arrays of small cavities. Using a foam as the material of the unstable platform, an IMU is mounted on a rigid base plate which is connected to one or more rigid longitudinal bodies that radiate from the center of the base plate to cover a large area. The IMU/longitudinal bodies assembly is situated the center of the foam. When an individual stands on the flat platform to monitor the balance index, his body weight will exert a downward force from the center of gravity (COG) of his body on the longitudinal bodies. The movement of the longitudinal bodies will be transmitted to move the IMU. COG movements and location of the downward COG projection of the test subject while keeping his balance on the flat platform is measured as IMU signals. The IMU signals is used to determine the balance index of the test subject. The IMU/longitudinal bodies assembly can either be placed on the top and being exposed on the surface of the platform or can be buried just below the surface and completely concealed. Therefore, this device can be used to measure balance index or as a platform for exercise or game when it is placed with the IMU location facing downward to the ground. The performance of the present apparatus is validated by measuring the movement of an IMU placed on the surface of the foam with and without rigid rods as described when a person is standing on it while trying to keep balance. FIGS. 5a-5b clearly indicates the effectiveness in enhancing the sensitivity without sacrificing the performance.

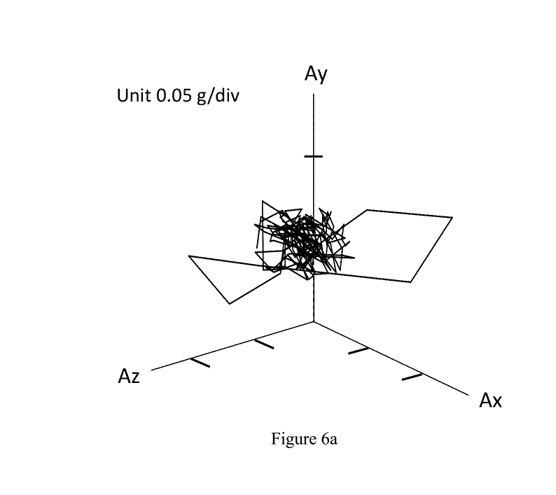

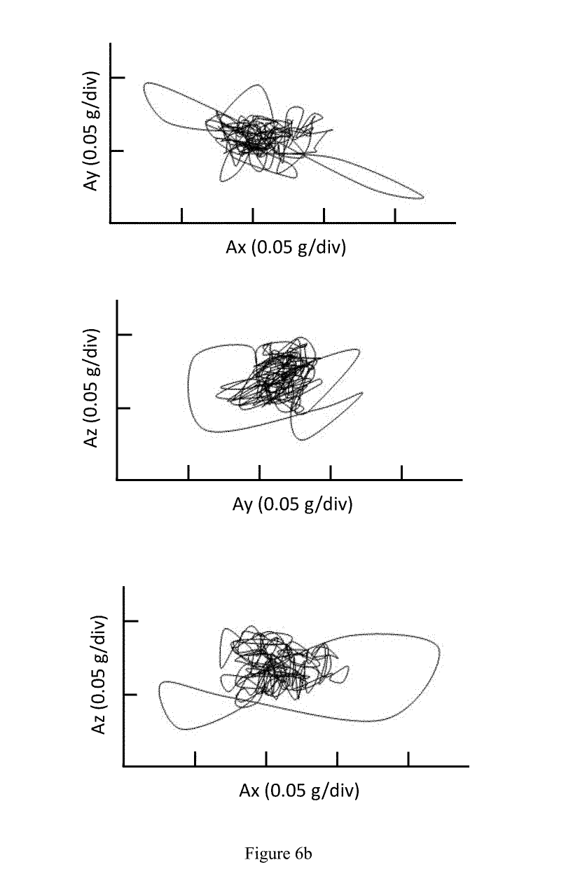

A tri-axial IMU is embedded at the center of the platform to record acceleration along all three axes when the subject is moving to keep balance. FIGS. 6a-6b depicts the 3D and 2D acceleration trajectory recorded at 100 Hz data rate. Information relevant to balance can be extracted from the IMU data is elaborated below.

There are a number of options for data collection and analysis. Data collected by the IMU can be stored on board for analysis or transmitted to a computer or a handset where data analysis and storage take place. Transmitting the measured data and data analysis on a computer system can reduce the hardware cost and size. In one embodiment of the present invention the data acquisition and data analysis of said apparatus are carried out with a computing device which is separate from the unstable platform. In this embodiment, the computing device and the flat unstable platform connected via wired or wireless data connection.

Methodology for Data Extraction:

A Number of Algorithms Can be Used to Extract Information Relevant to Balance Ability of a Test Subject from the Measured IMU Data.

Balance Index:

Balance index is represented by a quantity that is related to the effort spent to keep balanced and avoid falling. A large value balance index indicates a higher level of difficulty to keep balanced.

The following is a way to extract information on balance index from the measured IMU data.

Gait Power

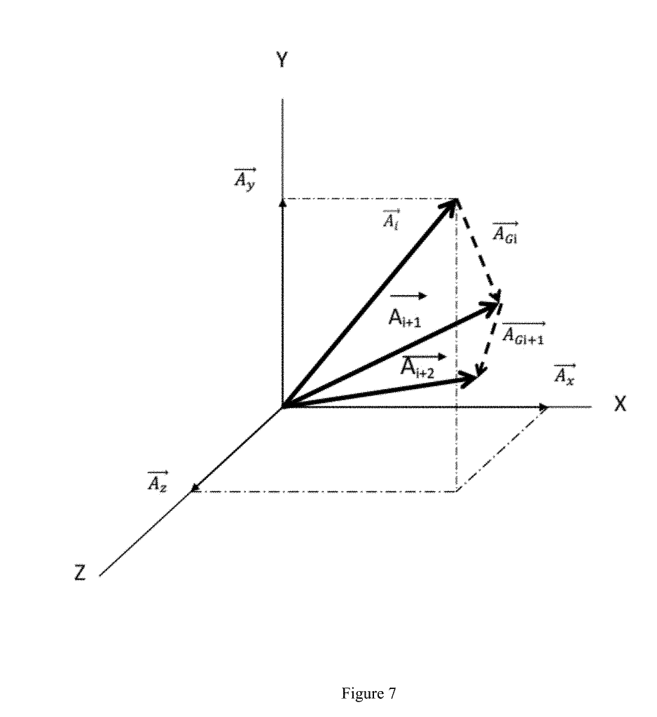

A data point, measured at time tᵢ, is represented by an acceleration vector \vec{Aᵢ} of the Center of Gravity of the subject under test, or known as gait force vector, as shown in FIG. 7. The component vectors along the three major axes of the Cartesian coordinate system defined by the subject under test are: \vec{Aₓ}, \vec{A_{y}} and \vec{A_{z}} that designate the acceleration along side to side, top to bottom, and front to back orientations, respectively. Their magnitudes are xᵢ, yᵢ and zᵢ. A subsequent measurement, made after a duration Δt, is represented by vector \vec{Aᵢ₊}. Its component magnitudes are xᵢ₊ᵢ, yᵢ₊ᵢ and zᵢ₊ᵢ. The movement of the COG during this time can be described by the vector \vec{A_{Gi}}=\vec{Aᵢ₊₁}-\vec{Aᵢ}. The magnitude of this vector can be calculated to be {square root over (xᵢ₊₁−xᵢ)²+(yᵢ₊₁−yᵢ)²+(zᵢ₊₁−zᵢ)²)}. The process continues to form a trajectory in the 3D space shown in FIG. 6a and its 2D components in FIG. 6b. The total length of the trajectory, which can be obtained by summing all the trajectories, is proportional to the amount of energy spent during the time to maintain balance (referred to as Gait Energy in this application). Gait energy is then divided by the time duration to obtain an average value which is designated as Gait Power. The Gait Power is determined as the balance index. The higher the value, the more power expenditure is required to maintain balance. It is worthy to note that this approach accounts for movements in all three directions instead of just the planar motion used by existing instrument based on a static platform with force plate sensors. To take full advantage of this above formulation, the platform described in this invention is mounted on an unstable wobbly support base that delivers movements in all three dimensions during measurements. A balance index is derived from the normalized value of Gait Power.

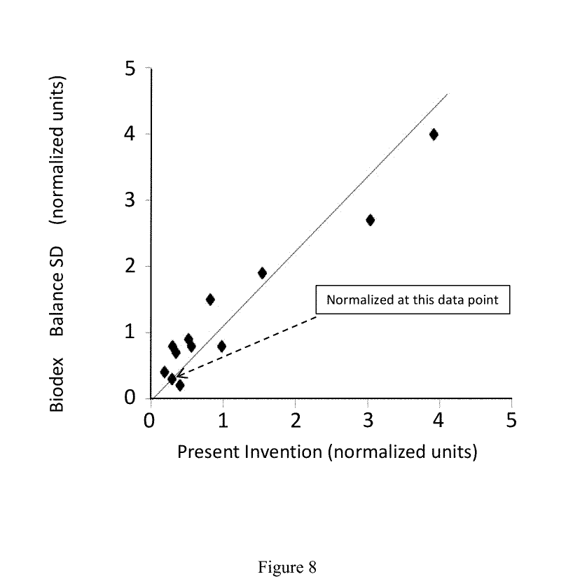

Results presented below are measured on a platform that consists of two parallel platforms connected by six identical compression springs as the elastic supports. Each spring have a spring constant of 80 Newton/cm. They are equally spaced as a 26 cm diameter circle from the center of the platform. Balance results of 12 individuals are measured on this embodiment of the present invention and on a Biodex Balance System SD under the same condition for comparison. FIG. 8 compares the results measured on the two instruments. When the data are normalized at the lowest balance index, the rest show a linear correlation over a broad dynamic range, thus validating the performance of the present invention. By using the same formulation, one can also separate the Gait Force vectors into orthogonal components to improve the accuracy of the balance assessment, especially the movements along the lateral-medial (i.e. side to side) and anterior-posterior (i.e. forward and backward) orientations.

Balance Anisotropy and Fall Risk Assessment.

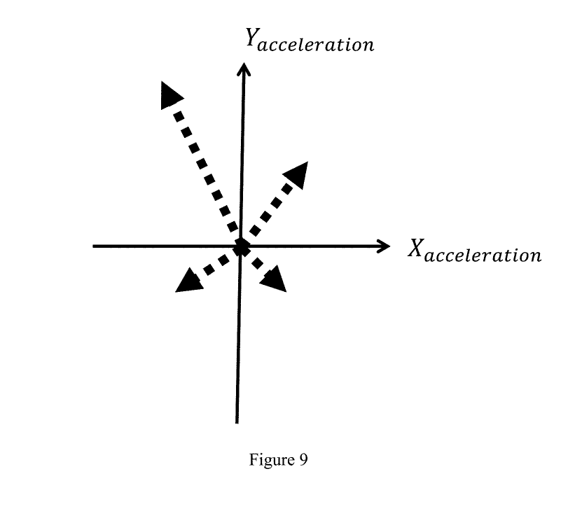

Typically, the biomechanics of balance of a human subject is anisotropic along different orientations. The direction and relative magnitude of the average acceleration vectors in each quadrant of the 12 test individuals is shown in FIG. 9. The information can be used to predict the most probable direction of falling. The quadrants can be represented by the front facing direction, the back direction, the left direction and the right direction of a human subject being measured on an unstable platform.

Postural Deviation

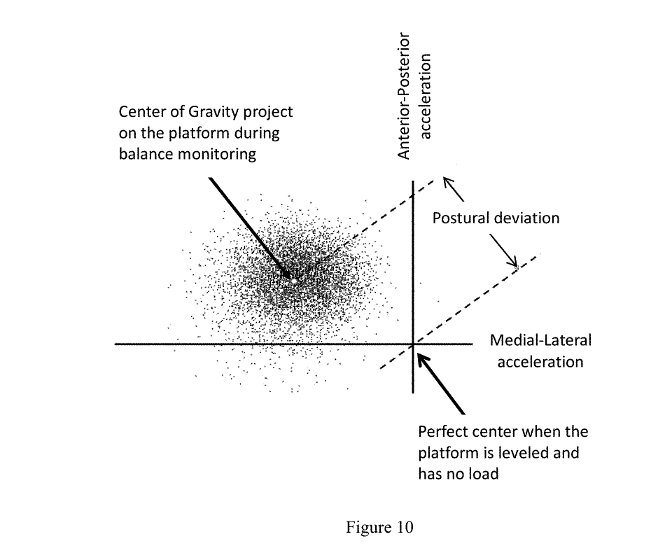

In the present application, the postural deviation is defined as the deviation of test subject's COG projection on the platform from the COG when the platform is leveled and unloaded. The postural deviation can be measured by examining the center of mass of the distribution of acceleration. FIG. 10 shows the spatial distribution of the location of the acceleration vectors in the horizontal plane at every time increment during the measurement. The tip of each vector is represented by a dot. The displacement between the center of mass of this set of data and the true center when the platform is leveled and unloaded is a measure of the postural deviation. The result shows not only the magnitude but also the direction of deviation.

Determination of Reflex Time

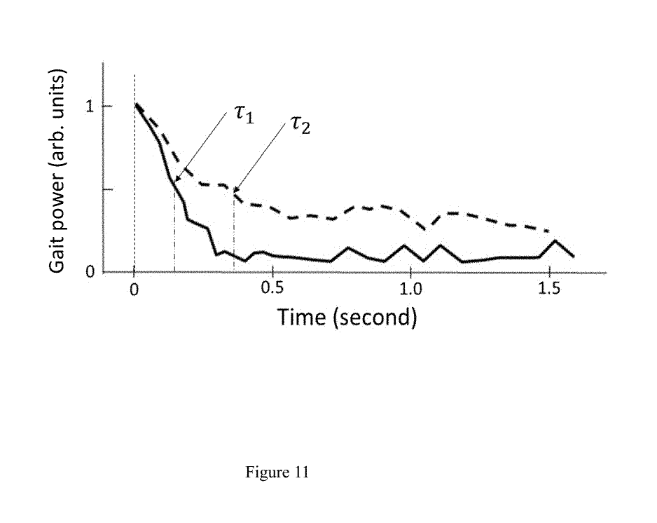

Unlike the existing static balance platform mounted on force plates, this invention uses an elastic support mounted platform that is dynamic and can interact with the subject under test. This dynamic interaction provided by the configuration of the present apparatus can be used to measure the recovery time after initial mechanical excitation by recording the change of the gait force with time. This measurement will yield information about the reflex of the human subject. FIG. 11 shows the comparison of recovery time between two individuals with different balance index of 15 and 36, respectively. The one with a higher balance index/gait power has a longer recovery time.

Measurement Procedure

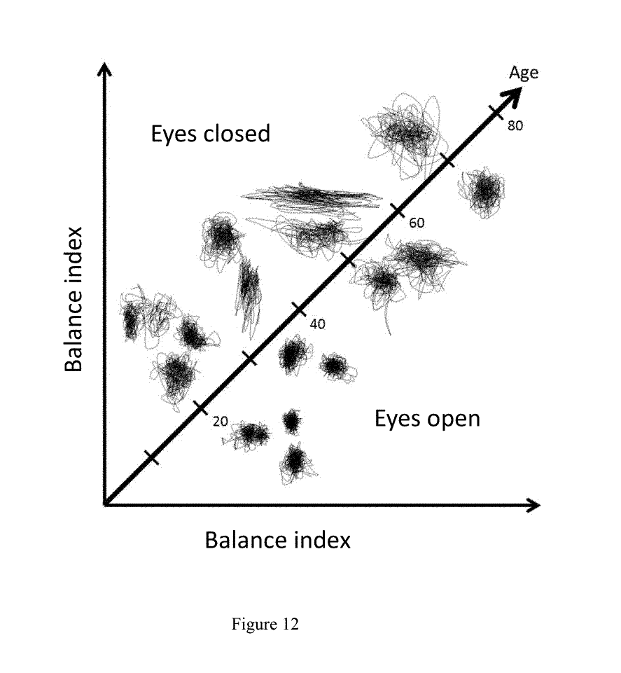

The present apparatus is placed on leveled ground with its IMU turned on to establish the true center of the system without a load. The human subject will then step onto the platform and maintain balance. For existing device with a static platform embedded with force plates, the human subject must shift his COG from one location to another predesignated location. The person must “see” his COG location on the display screen and use it as a visual guide. This requirement makes the assessment of the balance without visual sensor feedback difficult and inaccurate. However, in present invention, the test subject senses the imbalance due to his continuous dynamic interaction with the unstable wobbly elastic support mounted platform even with eyes closed and make adjustment in his COG accordingly. This unique feature enables to assess the vestibular effect on balance independently. This is a major difference and advantage between the present invention and existing balance measuring apparatus based on a static platform with force plate sensors. The ability to study the effect of visual and vestibular independently is a powerful tool. As shown in FIG. 12, the loss of balance due to vestibular sensor degradation occurring at a much faster rate than visual sensor with age. Results are shown in FIG. 12. Accordingly, the present invention provides an alternative way to measure balance ability that is independent of visual sensory feedback of the test subject.

In addition, the dynamic platform action coupled with acceleration measurement also requires a shorter measurement time than the force plate based apparatus. A 10-20 seconds long measurement will be sufficient.

Data analysis is carried out in real time starting from the onset of the measurement. Balance index is calculated from the starting time for a preselected duration. After completing the analysis of the first set of data, the second set will start immediately. This process will continue until the measurement is complete. The data set that shows the best balance index will be used. This practice will eliminate the need for data range selection and simplify the measurement process. Further information such as balance anisotropy and fall risk assessment can be calculated either in real time or on-demand.

It should also point out that this invention measures the movement of the COG of the human subject in all three dimensions which is different from measurement based on force plate sensors and offers a wealthier amount of information than the use of force plate based device that provides 2D information only.

Finally, with this data analysis and presentation format, the flat platform of the present invention can be further integrated with a weight scale or exercise pad to be used as a multi-functional personal health monitoring equipment. It can also be integrated with an elastomer foam that is used for balance exercise and training.

Games and Balance Training Tool

The combination of a simple dynamic platform with embedded IMU can also be used for interactive games and rehabilitation exercise to improve balancing skill. An example is to move one's body to guide the projection of the COG from one location to another predesignated spot, or to guide it through a predesignated meandering path, or simulate a sport even such as surfing and skiing. A score will be assigned on the basis of accuracy and time spent to accomplish the task. The complexity can be varied to change the level of difficulty. Not only such exercise can improve one's balancing skill, it also blends the otherwise boring physical exercise with the element of fun and challenge of playing games. In comparison to similar routines on static platform with force plates, the dynamic elastic support mounted platform will present much more realistic experience in simulating the terrain surface topography due to the dynamic interaction between the body movement and the 3D response of the elastic support mounted platform. Comparing with some high end commercial product such as skiing simulator, this invention offers the advantage in simplicity, compact size, low cost, and the flexibility to be converted into different form of exercise and games.

The device can also record the exercise routine in the form of acceleration along three axes. Together with the balance index measured on the same instrument, it can be used to assess the effectiveness of exercise routine.

The electronic embodiments disclosed herein may be implemented using general purpose or specialized computing devices, computer processors, or electronic circuitries including but not limited to application specific integrated circuits (ASIC), field programmable gate arrays (FPGA), and other programmable logic devices configured or programmed according to the teachings of the present disclosure. Computer instructions or software codes running in the general purpose or specialized computing devices, computer processors, or programmable logic devices can readily be prepared by practitioners skilled in the software or electronic art based on the teachings of the present disclosure.

The electronic embodiments include computer storage media having computer instructions or software codes stored therein which can be used to program computers or microprocessors to perform any of the processes of the present invention. The storage media can include, but are not limited to, floppy disks, optical discs, Blu-ray Disc, DVD, CD-ROMs, and magneto-optical disks, ROMs, RAMs, flash memory devices, or any type of media or devices suitable for storing instructions, codes, and/or data.

### INDUSTRIAL APPLICABILITY

The objective of the presently claimed invention is to provide method and apparatus for measuring the balance ability; postural deviation, reflex and fall risk assessment of a human subject. In particular, the present invention comprises an elastic support mounted platform, a IMU and data analysis software. The present apparatus can further be integrated with other measuring devices, such as a regular bathroom weight scale to be used as a multifunctional personal health monitoring device. The dynamic response from the platform also simulates realistic experience in moving over rough terrain with different topography; it can be applied to create interactive action for gaming and balance training.

1. A method for measuring a balance index of a human subject comprising providing a flat platform mounted on one or more elastic supports to provide a wobbly surface for the subject to stand on, the platform comprises an inertia measurement unit (IMU);
placing the subject onto the flat platform;
measuring center of gravity (COG) of the subject while the subject balances on the platform;
measuring movements of COG of said human subject in three orthogonal vectors over a time interval to obtain a COG trajectory in a three-dimensional space over the time interval;
summing over the COG trajectory of said human subject in three-dimensional space to obtain a gait energy over said time interval;
dividing said gait energy over said time interval to obtain a gait power value; and
normalizing said gait power value to determine as a measure of balance index for said human subject.

2. A method for measuring balance anisotropy and assessing risk of falling for a human subject comprising
measuring relative magnitude of the balance index in four orthogonal directions of said human subject, wherein the balance index is measured according to the method of claim 1 and the four orthogonal direction consist of front facing direction, back direction, left direction and right direction of said human subject.

3. A method for measuring postural deviation of a human subject comprising
measuring COG trajectory of the human subject on a flat platform according to claim 1 and obtaining a deviation of the COG trajectory of the human subject standing on the flat platform from COG of an unloaded flat platform.

4. The method according to claim 1, wherein the IMU measures acceleration in all three orthogonal directions, and wherein the elastic supports are selected from compression springs, extension springs, leaf springs, magnetic repulsion, elastomer balls and cylinders, closed cell high density elastomer foam cushion, inflated rubber ball, inflated rubber cushion, floats on fluid, suspended platform and a combination thereof.

5. The method according to claim 1, wherein said flat platform is a closed cell high density elastomer foam pad having a rigid base plate at the centre of the platform and the IMU is mounted on a rigid base plate, wherein said rigid base plate is connected to at least one rigid longitudinal body that radiate from the center of the rigid base plate.

6. The method according to claim 5, wherein said flat platform further comprises arrays of cavities.

7. The method according to claim 6, wherein the IMU, the rigid base plate and the at least one rigid longitudinal body are embedded inside the flat platform.

8. The method according to claim 5, wherein the IMU is a tri-axial accelerometer.

9. A balance index measuring apparatus comprising a flat platform mounted on one or more elastic supports to provide a wobbly surface for a human subject to stand on, the platform comprises an IMU.

10. The apparatus according to claim 9, wherein the elastic supports are selected from the group consisting of compression springs, extension springs, leaf springs, magnetic repulsion, elastomer balls and cylinders, elastomer foam cushion, inflated rubber ball, inflated rubber cushion, floats on fluid and suspended platform and a combination thereof.

11. The apparatus according to claim 9, wherein said flat platform is an elastomer foam pad having a rigid base plate at the center of the platform and the IMU is mounted on the rigid base plate, wherein said base plate is connected to at least one rigid longitudinal body that radiate from the center of the base plate.

12. The apparatus according to claim 11 wherein the IMU, the rigid base plate and the at least one rigid longitudinal body are embedded inside the platform.

13. The apparatus according to claim 12, wherein the IMU is a tri-axial accelerometer.

14. The apparatus according to claim 9 further comprises a computing device, wherein the computing device is in electronic connection with the platform via wired or wireless data connection.

15. The apparatus according to claim 9 further comprises a weight scale in connection with the flat platform.
